(19) United States
(12) Patent Application Publication
(10) Pub. No.: US 2022/0187233 A1
(43) Pub. Date: Jun. 16, 2022

(54) GAS ADSORBENT, GAS ADSORBING DEVICE, AND GAS SENSOR

(71) Applicant: PANASONIC CORPORATION, Osaka (JP)

(72) Inventor: Atsushi SHUNORI, Osaka (JP)

(21) Appl. No.: 17/599,466

(22) PCT Filed: Mar. 26, 2020
(86) PCT No.: PCT/JP2020/013692

§ 371 Date: Sep. 28, 2021

(30) Foreign Application Priority Data
Mar. 28, 2019 (JP) 2019-065101

(51) Int. Cl. G01N 27/12

(52) U.S. Cl. CPC G01N 27/126

(57) ABSTRACT

A gas adsorbent includes a plurality of adsorbent particles. The plurality of adsorbent particles are aggregated together to form a porous structure. Each of the adsorbent particles includes: an insulating particle; and a plurality of conductive particles and an organic material, all of which adhere to a surface of the insulating particle.

### TECHNICAL FIELD

The present disclosure generally relates to a gas adsorbent, a gas adsorbing device, and a gas sensor, and more particularly relates to a gas adsorbent containing an organic material and conductive particles, a gas adsorbing device including the gas adsorbent, and a gas sensor including either the gas adsorbent or the gas adsorbing device.

### BACKGROUND ART

Gas sensors, containing a gas-adsorbing organic material and conductive particles dispersed in the organic material, have been provided. For example, Patent Literature 1 discloses a chemiresistor including: an electrically insulating base member with a pair of electrodes arranged in a circularly shaped geometry in parallel with each other; a chemically sensitive polymer in contact with the pair of electrodes; and carbon particles dispersed in the chemically sensitive polymer. In this chemiresistor, when the chemically sensitive polymer adsorbs a volatile organic compound in a gas, for example, its electrical resistance value varies. Using this chemiresistor allows the volatile organic compound in the gas to be detected based on the variation in the electrical resistance value of the chemiresistor.

### CITATION LIST

### Patent Literature

Patent Literature 1: U.S. Pat. No. 7,189,360 B1

### SUMMARY OF INVENTION

The problem to be overcome by the present disclosure is to provide a gas adsorbent containing an organic material and a plurality of conductive particles and allowing an electrical resistance value to vary responsively upon exposure to a gas, a gas adsorbing device including the gas adsorbent, and a gas sensor including either the gas adsorbent or the gas adsorbing device.

A gas adsorbent according to an aspect of the present disclosure includes a plurality of adsorbent particles. The plurality of adsorbent particles are aggregated together to form a porous structure. Each of the plurality of adsorbent particles includes: an insulating particle; and a plurality of conductive particles and an organic material, all of which adhere to a surface of the insulating particle.

A gas adsorbing device according to another aspect of the present disclosure includes: the gas adsorbent described above; and a base member. Each of the plurality of adsorbent particles of the gas adsorbent includes: the insulating particle; a first coating layer formed of the plurality of conductive particles and coating the surface of the insulating particle continuously; and a second coating layer formed of the organic material and coating a surface of the first coating layer continuously. The porous structure is formed by connecting the plurality of adsorbent particles with each other continuously and creating a void surrounded with adjacent ones of the plurality of adsorbent particles. The gas adsorbent is in contact with the base member on at least one of the first coating layer or the second coating layer.

A gas sensor according to still another aspect of the present disclosure includes: either the gas adsorbent described above or the gas adsorbing device described above; and an electrode electrically connected to the gas adsorbent.

### BRIEF DESCRIPTION OF DRAWINGS

FIG. 1 is a schematic cross-sectional view of a gas adsorbent, a gas adsorbing device, and a gas sensor according to an exemplary embodiment of the present disclosure;

FIG. 2 is a schematic plan view of a gas sensor under test, which was used in an example;

FIG. 3 is a graph showing variation rates of electrical resistance values which were measured in an example with respect to Samples #1, #2, and #3 when nonanal was adsorbed thereto;

FIG. 4A is a scanning electron microscope (SEM) micrograph showing a cross section of Sample #7 of the example;

FIG. 4B is an SEM micrograph showing a cross section of Sample #8 of the example;

FIG. 4C is an SEM micrograph showing a cross section of Sample #3 of the example;

FIG. 5A is an SEM micrograph showing a surface of Sample #7 of the example;

FIG. 5B is an SEM micrograph showing a surface of Sample #8 of the example;

FIG. 5C is an SEM micrograph showing a surface of Sample #3 of the example;

FIG. 6 is a graph showing how the variation rate of the electrical resistance value changed with time when measured in the example with respect to Samples #3-8 with benzaldehyde adsorbed thereto; and

FIG. 7 is a graph showing how the variation rate of the electrical resistance value changed when measured in the example with respect to Samples #3-8 with benzaldehyde adsorbed thereto.

### DESCRIPTION OF EMBODIMENTS

First, it will be described exactly how the present inventors conceived the idea of the present disclosure.

If a chemical substance in a gas is absorbed into a gas adsorbent, containing an organic material and conductive particles dispersed in the organic material, by exposing the gas adsorbent to the gas, then the electrical resistance value of the gas adsorbent varies. The electrical resistance value varies partly and presumably because the organic material adsorbs the chemical substance and expands, thus changing the gap distance between the conductive particles in the gas adsorbent. The chemical substance in the gas may be detected based on such a variation in the electrical resistance value of the gas adsorbent. That is to say, the chemical substance in the gas may be detected by using a gas sensor including such a gas adsorbent.

In a situation where the gas adsorbent is exposed to a gas, the more significantly and the more quickly the electrical resistance value of the gas adsorbent varies, the more quickly and the more accurately the chemical substance may be detected.

However, there has been a limit to the performance enhancement of the gas adsorbent by, for example, changing the combination of the organic material and conductive particles selected.

Thus, the present inventors carried out extensive research to develop a gas adsorbent, of which the electrical resistance value would vary responsively upon exposure to a gas containing a chemical substance. As a result of the extensive research and development, the present inventors conceived the idea of the present disclosure.

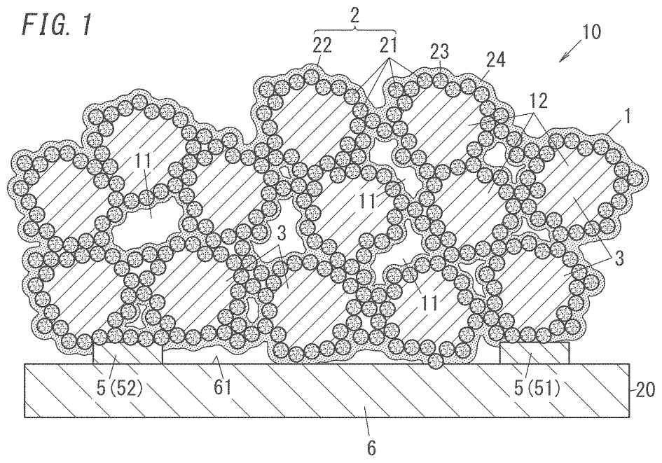

Next, an exemplary embodiment of the present disclosure will be described with reference to FIG. 1.

A gas adsorbent 1 according to this embodiment includes a plurality of adsorbent particles 12. The adsorbent particles 12 are particles having gas adsorptivity. As used herein, the “gas adsorptivity” refers to the property of adsorbing a chemical substance contained in a gas upon exposure to the gas. Examples of the chemical substances include volatile organic compounds and inorganic compounds. Examples of the volatile organic compounds include ketones, amines, alcohols, aromatic hydrocarbons, aldehydes, esters, organic acids, methyl mercaptans, and disulfides. Examples of the inorganic compounds include hydrogen sulfide, sulfur dioxide, and carbon disulfide. The adsorbent particles 12 preferably have the property of adsorbing at least one type of chemical substance. It may be determined based on common technical knowledge whether the adsorbent particles 12 have gas adsorptivity. For example, if a chemical substance derived from a gas is detected when the adsorbent particles 12 are exposed to the gas and then analyzed with a gas chromatograph mass spectrometer, it may be determined that the adsorbent particles 12 should have gas adsorptivity. The adsorbent particles 12 preferably have the property of adsorbing at least one type of volatile organic compound.

The adsorbent particles 12 are aggregated together to form a porous structure. Each of the adsorbent particles 12 includes an insulating particle 3 and a conductive particle 21 and an organic material 22, both of which adhere to the surface of the insulating particle 3.

Stated otherwise, it can be said that the gas adsorbent 1 includes the insulating particle 3 and an adsorbing portion 2. In that case, the adsorbing portion 2 includes the conductive particle 21 and the organic material 22. In the adsorbing portion 2, the conductive particles 21 may be dispersed in the organic material 22, for example. In the gas adsorbent 1, the insulating particles 3, each having a surface to which the adsorbing portion 2 adheres, are aggregated together to form a porous structure.

Specifically, each of the adsorbent particles 12 may include, for example: the insulating particle 3; a first coating layer 23 formed of the conductive particle(s) 21 and coating the surface of the insulating particle 3 continuously; and a second coating layer 24 formed of the organic material 22 and coating the surface of the first coating layer 23 continuously. The porous structure of the gas adsorbent 1 is formed by connecting the adsorbent particles 12 with each other continuously and creating voids 11 surrounded with adjacent ones of the adsorbent particles 12. That is to say, each of the voids 11 in the porous structure is surrounded with the adsorbent particles 12. In this case, the adsorbing portion 2 is formed by aggregating the adsorbent particles 12 together such that the respective first coating layers 23 and second coating layers 24 of the adsorbent particles 12 are integrated together.

A gas adsorbing device 20 according to this embodiment includes the gas adsorbent 1 and a base member 6. For example, the gas adsorbent 1 is in contact with the base member 6 on at least one of the first coating layer 23 or the second coating layer 24.

Specifically, the conductive particle(s) 21 and the organic material 22 may adhere, for example, in the shape of a film, to the surface of the insulating particle 3. Stated otherwise, it can also be said that the adsorbing portion 2 has the shape of a film which adheres to the surface of the insulating particle 3 and thereby covers the insulating particle 3. In this case, the conductive particle(s) 21 and the organic material 22 (i.e., the adsorbing portion 2) may cover the insulating particle 3 either entirely or only partially, whichever is appropriate. In the gas adsorbent 1, if the gap between adjacent insulating particles 3 is narrow enough or if the insulating particles 3 are in contact with each other, then the respective conductive particles 21 and organic material 22 (i.e., the adsorbing portions 2) adhering to the insulating particles 3 tend to be bonded and integrated together. On the other hand, if the gap between the adjacent insulating particles 3 is wide enough, then a void 11 tends to be formed between the particles 3. Thus, the insulating particles 3, each having a surface to which the conductive particle(s) 21 and the organic material 22 (i.e., the adsorbing portion 2) adhere, are aggregated together to form a porous structure. Note that it depends on the individual situation, and is difficult to explicitly define, how narrow the gap should be to increase the chances of the adsorbing portions 2 being bonded together and how wide the gap should be to increase the chances of creating the void 11.

When the gas adsorbent 1 according to this embodiment is exposed to a gas, the organic material 22 adsorbs a chemical substance in the gas, and therefore, the electrical resistance value of the gas adsorbent 1 varies accordingly.

This embodiment increases, when the gas adsorbent 1 is exposed to a gas, the chances of the electrical resistance value of the gas adsorbent 1 varying quickly. That is to say, this embodiment facilitates improving the responsivity of the gas adsorbent 1. This should be partly because the porosity of the gas adsorbent 1 should allow the gas to enter the voids 11 in the gas adsorbent 1 more easily (i.e., increases the gas permeability of the gas adsorbent 1) to the point that the gas adsorbent 1 may adsorb a chemical substance in the gas efficiently.

The organic material 22 preferably has gas adsorptivity. As used herein, the “gas adsorptivity” refers to the property of adsorbing a chemical substance contained in a gas upon exposure to the gas. Examples of the chemical substances include volatile organic compounds and inorganic compounds. Examples of the volatile organic compounds include ketones, amines, alcohols, aromatic hydrocarbons, aldehydes, esters, organic acids, methyl mercaptans, and disulfides. Examples of the inorganic compounds include hydrogen sulfide, sulfur dioxide, and carbon disulfide. The organic material 22 preferably has the property of adsorbing at least one type of chemical substance. It may be determined based on common technical knowledge whether the organic material 22 has gas adsorptivity. For example, if a chemical substance derived from a gas is detected when the organic material 22 is exposed to the gas and then analyzed with a gas chromatograph mass spectrometer, it may be determined that the organic material 22 should have gas adsorptivity. The organic material 22 preferably has the property of adsorbing at least one type of volatile organic compound.

The organic material 22 is selected according to, for example, the type of the chemical substance that the gas adsorbent 1 needs to adsorb and the type of the conductive particles 21 in the gas adsorbent 1. The organic material 22 includes at least one type of material selected from the group consisting of polymers and low molecular weight compounds. Among other things, the organic material 22 preferably includes a polymer. If the organic material 22 includes a polymer, the gas adsorbent 1 may have heat resistance.

Examples of preferred organic materials 22 include materials which are commercially available as stationary phases of columns in gas chromatographs. More specifically, the organic material 22 includes at least one material selected from the group consisting of, for example, polyalkylene glycols, polyesters, silicones, glycerols, nitriles, dicarboxylic acid monoesters, and aliphatic amines. In this case, the organic material 22 may easily adsorb a chemical substance in the gas, particularly a volatile organic compound.

Polyalkylene glycols include, for example, polyethylene glycol (with a heat resisting temperature of 170° C.). Polyesters include, for example, at least one material selected from the group consisting of poly(diethylene glycol adipate) and poly(ethylene succinate). Silicones include, for example, at least one material selected from the group consisting of dimethyl silicone, phenylmethyl silicone, trifluoropropylmethyl silicone, and cyanosilicone (with a heat resisting temperature of 275° C.). The glycerols include, for example, diglycerol (with a heat resisting temperature of 150° C.). Nitriles include, for example, at least one material selected from the group consisting of N,N-bis(2-cyanoethyl) formamide (with a heat resisting temperature of 125° C.) and 1,2,3-tris(2-cyanoethoxy) propane (with a heat resisting temperature of 150° C.). Dicarboxylic acid monoesters include, for example, at least one material selected from the group consisting of nitroterephthalic acid modified polyethylene glycol (with a heat resisting temperature of 275° C.) and diethylene glycol succinate (with a heat resisting temperature of 225° C.). Aliphatic amines include, for example, tetrahydroxyethyl-ethylenediamine (with a heat resisting temperature of 125° C.).

The conductive particle 21 includes, for example, at least one material selected from the group consisting of carbon materials, conductive polymers, metals, metal oxides, semiconductors, superconductors, and complex compounds. The carbon material includes, for example, at least one material selected from the group consisting of carbon black, graphite, coke, carbon nanotubes, graphene, and fullerenes. The conductive polymer includes, for example, at least one material selected from the group consisting of polyaniline, polythiophene, polypyrrole, and polyacetylene. The metal includes, for example, at least one material selected from the group consisting of silver, gold, copper, platinum, and aluminum. The metal oxide includes, for example, at least one material selected from the group consisting of indium oxide, tin oxide, tungsten oxide, zinc oxide, and titanium oxide. The semiconductor includes, for example, at least one material selected from the group consisting of silicon, gallium arsenide, indium phosphide, and molybdenum sulfide. The superconductor includes, for example, at least one material selected from the group consisting of YBa₂Cu₃O₇ and T₁₂Ba₂Ca₂Cu₃O₁₀. The complex compound includes, for example, at least one material selected from the group consisting of: a complex compound of tetramethylparaphenylenediamine and chloranil; a complex compound of tetracyanoquinodimethane and an alkali metal; a complex compound of tetrathiafulvalene and halogen; a complex compound of iridium and halocarbonyl compound; and tetracyano platinum.

The conductive particle 21 preferably includes a carbon material. Among other things, the conductive particle 21 particularly preferably includes carbon black. If the conductive particle 21 includes a carbon material (such as carbon black, in particular), the electrical resistance value of the gas adsorbent 1 may vary particularly responsively upon exposure to a gas.

The mean particle size of the conductive particles 21 is preferably less than 50 nm, more preferably equal to or less than 44 nm, and even more preferably equal to or less than 30 nm. The mean particle size is preferably equal to or less than 25 nm, more preferably equal to or less than 20 nm, and particularly preferably equal to or less than 15 nm. The smaller the mean particle size of the conductive particles 21 is, the more significantly the variation rate of the resistance value of the gas adsorbent 1 increases when the gas adsorbent 1 adsorbs a chemical substance (i.e., the higher the sensitivity of the gas adsorbent 1 becomes).

According to this embodiment, even though the mean particle size of the conductive particles 21 is as small as described above, the gas adsorbent 1 is still allowed to have the porous structure by using the insulating particle 3. That is to say, even if the void 11 is not created easily since the mean particle size of the conductive particles 21 is small, the void 11 may still be created and the porous structure may still be formed by adjusting the particle size of the insulating particles 3.

The lower limit of the mean particle size of the conductive particles 21 in the adsorbing portion 2 is not defined to be any particular value. Nevertheless, to increase the degree of homogeneity of the gas adsorbent 1 by reducing the chances of the conductive particles 21 aggregating together, the mean particle size is preferably equal to or greater than 5 nm and more preferably equal to or greater than 10 nm.

Note that the mean particle size of the conductive particles 21 is a number-based arithmetic mean of the particle sizes, obtained based on an electron microscope micrograph, of the conductive particles 21. Specifically, the electron microscope micrograph is subjected to image processing to derive the respective areas of conductive particles 21 appearing on the electron microscope micrograph. Next, based on these areas of the conductive particles 21, the diameter of each of the conductive particles 21 when the particle is transformed into a perfect circle is calculated. Then, the average of these diameters is calculated to obtain the mean particle size.

Note that the shape of the conductive particles 21 is not limited to any particular one. Thus, the conductive particles 21 may have a spherical, ellipsoidal, crushed, or scale shape, whatever is appropriate.

The insulating particle 3 includes, for example, at least one of a resin material with electrical insulation properties or an inorganic material with electrical insulation properties. The resin material having electrical insulation properties of the insulating particle 3 includes at least one material selected from the group consisting of, for example, silicone, acrylic resins, melamine resins, epoxy resins, polylactic acid resins, ethyl cellulose resins, and polyether sulfone resins. The inorganic material with electrical insulation properties includes at least one material selected from the group consisting of, for example, silica, aluminum oxide, zinc oxide, tin oxide, titanium oxide, copper oxide, tungsten oxide, iron zirconia oxide, magnesium oxide, yttrium oxide, barium titanate, hydroxyapatite, titanium carbide, and aluminum nitride.

Note that the shape of the insulating particles 3 is not limited to any particular one. Thus, the insulating particles 3 may have a spherical, ellipsoidal, crushed, or scale shape, whatever is appropriate.

The mean particle size of the insulating particles 3 preferably falls within the range from 50 nm to 2000 nm. This increases the chances of a void 11 of an appropriate size for a gas to permeate being created in the gas adsorbent 1, thus improving the responsivity of the gas adsorbent 1 particularly significantly. The mean particle size of the insulating particles 3 is more preferably equal to or greater than 100 nm and less than 1500 nm. This facilitates improving the sensitivity of the gas adsorbent 1. This is presumably because if the mean particle size is less than 1500 nm, the size of the void 11 would not become too large, thus increasing the specific surface area of the void 11.

Note that the mean particle size of the insulating particles 3 is a numerical value calculated based on a particle size distribution obtained by dynamic light scattering method. As a measuring device for measuring the mean particle size, Zetasizer Nano ZS90 manufactured by Malvern Panalytical, for example, may be used.

The mean particle size of the insulating particles 3 is preferably larger than the mean particle size of the conductive particles 21. This increases the chances of the conductive particles 21 adhering to the surface of the insulating particle 3, thus facilitating forming the first coating layer 23. Consequently, a porous structure with voids 11 is formed more easily.

In particular, the mean particle size of the insulating particles 3 is preferably three times or more as large as the mean particle size of the conductive particles 21. This increases the chances of creating voids 11 and allowing each of the voids 11 to have an appropriate size for the gas to pass therethrough. The mean particle size of the insulating particles 3 is more preferably five times or more as large as the mean particle size of the conductive particles 21. The upper limit of the ratio of the mean particle size of the insulating particles 3 to the mean particle size of the conductive particles 21 is not limited to any particular value but may be 100 or less, for example.

The diameter of the voids 11 in the porous structure is preferably larger than the mean particle size of the conductive particles 21. This increases the chances of the gas adsorbent 1 having a porous structure and creating, in the gas adsorbent 1, voids 11 having an appropriate size for the gas to permeate therethrough. The diameter of the voids 11 may be determined by the following method. First, the gas adsorbent 1 is cut off to expose a cross section thereof. Next, the cross section thus exposed is polished and then observed through an electron microscope to capture a micrograph. Then, circles inscribed to the respective profiles of voids 11, appearing on the micrograph, are drawn and the diameter of the largest one of those inscribed circles is measured. For example, the diameter values of inscribed circles are measured with respect to ten voids 11 and the average of six intermediate ones of the diameter values, selected from the ten diameter values with the two largest ones and the two smallest ones excluded, is calculated and regarded as the diameter of the voids 11.

The respective contents of the organic compound, the conductive particles 21, and the insulating particles 3 that form the gas adsorbent 1 are set appropriately depending on, for example, the particle size of the conductive particles 21 and the particle size of the insulating particles 3 to allow the gas adsorbent 1 according to this embodiment to have the porous structure. In particular, the ratio by mass of the insulating particles 3, the conductive particles 21, and the organic material 22 is preferably close to 1:1:1. This increases the chances of the gas adsorbent 1 having the porous structure and creating, in the gas adsorbent 1, voids 11 of an appropriate size for the gas to permeate therethrough.

The gas adsorbent 1 preferably has the shape of a film. That is to say, the gas adsorbent 1 is preferably a porous film. This increases the specific surface area of the gas adsorbent 1, thus allowing the gas adsorbent 1 to adsorb a chemical substance in the gas more easily. The gas adsorbent 1 may have a thickness falling within the range from 0.1 μm to 10 μm, for example.

Next, a gas sensor 10 including either the gas adsorbent 1 or a gas adsorbing device 20 will be described. The gas sensor 10 includes either the gas adsorbent 1 or the gas adsorbing device 20; and an electrode 5 electrically connected to the gas adsorbent 1. Using this gas sensor 10 causes, when the gas adsorbent 1 is exposed to a gas containing a chemical substance, the gas adsorbent 1 to adsorb the chemical substance, thus causing a variation in the electrical resistance value of the gas adsorbent 1. The chemical substance may be detected based on this variation in electrical resistance value. This embodiment increases the chances of causing a variation in electrical resistance value when the gas adsorbent 1 is exposed to a gas as described above. Thus, the chemical substance in the gas may be detected accurately by using the gas sensor 10.

A specific example of the gas sensor 10 will be described with reference to FIG. 1. The gas sensor 10 includes the gas adsorbent 1 and the electrode 5. The electrode 5 includes a first electrode 51 and a second electrode 52. The gas sensor 10 further includes a base member 6. That is to say, the gas sensor 10 according to this specific example includes the gas adsorbing device 20 and the base member 6.

The base member 6 has electrical insulation properties. The base member 6 has one surface (hereinafter referred to as a “supporting surface 61”). On the supporting surface 61, arranged are the first electrode 51, the second electrode 52, and the gas adsorbent 1. The base member 6 may have the shape of a plate, which has thickness in the direction perpendicular to the supporting surface 61. The first electrode 51 and the second electrode 52 are spaced from each other in the direction perpendicular to the direction that the supporting surface 61 faces.

The gas adsorbent 1 is arranged on the supporting surface 61 of the base member 6. The gas adsorbent 1 is in contact with the base member 6 on at least one of the first coating layer 23 or the second coating layer 24 as described above. The gas adsorbent 1 covers the first electrode 51 and the second electrode 52. This brings the gas adsorbent 1 into contact with each of the first electrode 51 and the second electrode 52. Note that the electrical connection between the gas adsorbent 1 and each of the first electrode 51 and the second electrode 52 may be established by any structure. For example, the gas adsorbent 1 may be in contact with the first electrode 51 either entirely or only partially. Likewise, the gas adsorbent 1 may be in contact with the second electrode 52 either entirely or only partially.

When a voltage is applied between the first electrode 51 and the second electrode 52 of this gas sensor 10, an electric current flows through the gas adsorbent 1 in an amount corresponding to the voltage and the electrical resistance value of the gas adsorbent 1. This allows the electrical resistance value of the gas adsorbent 1 to be measured. A chemical substance may be detected based on the electrical resistance value. Optionally, the chemical substance may be detected based on the value of an electric current flowing between the first electrode 51 and the second electrode 52 in a state where constant voltage is applied between the first electrode 51 and the second electrode 52. Alternatively, the chemical substance may also be detected based on the magnitude of voltage drop between the first electrode 51 and the second electrode 52 in a state where a constant current is allowed to flow through the gas adsorbent 1. That is to say, the chemical substance may be detected based on a variation in some physical quantity (as an index) to be caused by a variation in the electrical resistance value of the gas adsorbent 1.

To fabricate this gas sensor 10, the first electrode 51 and the second electrode 52 may be provided on the supporting surface 61 of the base member 6, and then the gas adsorbent 1 is formed over the supporting surface 61, for example.

Next, a method of making the gas adsorbent 1 according to this embodiment will be described.

The gas adsorbent 1 may be made by preparing a mixed solution containing the organic material 22, the conductive particles 21, the insulating particles 3, and a solvent, forming a formed product out of the mixed solution, and then volatilizing the solvent out of the formed product.

The method of making the gas adsorbent 1 will be described more specifically. First, a mixed solution containing the organic material 22, the conductive particles 21, the insulating particles 3, and a solvent is prepared.

The organic material 22, the conductive particles 21, and the insulating particles 3 are as described above.

Any solvent may be used without limitation as long as the solvent may dissolve or disperse the organic material 22, may disperse the conductive particles 21 and the insulating particles 3, and may volatilize from the formed product. The solvent includes at least one component selected from the group consisting of, for example, dimethyl sulfoxide, dimethylformamide, toluene, chloroform, acetone, acetonitrile, methanol, ethanol, isopropanol, tetrahydrofuran, ethyl acetate, and butyl acetate.

Next, a formed product is formed out of the mixed solution. The formed product preferably has the shape of a film. In that case, the gas adsorbent 1 may be obtained in the shape of a film. Examples of the film shape include a film, a sheet, and a layer. The formed product may be formed by any method without limitation. The formed product may be formed by applying the mixed solution by an inkjet method, a dispensing method, or any other suitable method, for example. The formed product may have a thickness falling within the range from 0.1 μm to 10 μm, for example.

Next, the solvent is volatilized from the formed product. The solvent may be volatilized by any method without limitation. For example, the solvent may be volatilized from the formed product by subjecting the formed product to heat treatment. Alternatively, the solvent may also be volatilized from the formed product by placing the formed product under a reduced pressure. Optionally, the solvent may be volatilized from the formed product by subjecting the formed product to heat treatment under a reduced pressure. The temperature of the heat treatment may be set appropriately according to the type of the solvent so as to promote the volatilization of the solvent. The temperature of the heat treatment may fall within the range from 30° C. to 90° C., for example. In addition, the temperature of the heat treatment is preferably set to prevent, or at least retard, the pyrolysis of the organic material 22. Thus, the temperature of the heat treatment is preferably less than a temperature that is lower by 30° C. than the heat resisting temperature of the organic material 22. The duration of the heat treatment is preferably designed so as to volatilize either all or most of the solvent in the formed product through the heat treatment. The duration of the heat treatment may fall within the range from 10 minutes to 60 minutes, for example.

### EXAMPLES

Next, method and results of tests that the present inventors carried out with respect to the exemplary embodiment will be presented. Note that the method and results of the tests to be described below should not be construed as limiting the configuration of this embodiment.

1. Confirmation of Effect of Particle Size of Conductive Particles

Particles of carbon black with a mean particle size of 50 μm were provided as the conductive particles 21. As the solvent, dimethylformamide was provided. As the organic material 22, polyethylene glycol was provided.

The conductive particles 21 and the organic material 22 were added to the solvent and stirred up, thereby preparing a mixed solution including the conductive particles 21 at a concentration of 10 mg/ml and the organic material 22 at a concentration of 10 mg/ml.

Next, the mixed solution was applied by inkjet method to form a formed product in the shape of a film. This formed product was subjected to heat treatment at 50° C. for 20 minutes, thereby volatilizing the solvent from the formed product.

In this manner, Sample #1 was obtained as a gas adsorbent 1 containing no insulating particles 3. In addition, Sample #2 was obtained as another gas adsorbent 1 in the same way as Sample #1 except that the mean particle size of carbon black was changed into 44 nm. Furthermore, Sample #3 was obtained as still another gas adsorbent 1 in the same way as Sample #1 except that the mean particle size of carbon black was changed into 15 nm.

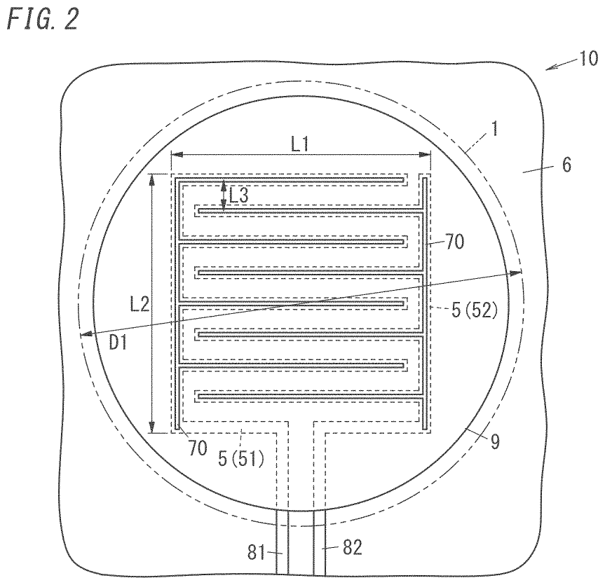

A gas sensor 10 under test was formed with each of these Samples #1-#3 used. A schematic structure of the gas sensor 10 under test is as shown in FIG. 2. In this gas sensor 10, a first electrode 51 and a second electrode 52 were arranged on a base member 6 with electrical insulation properties so as to form an interdigital electrode structure. A dimension L1 of the interdigital electrode structure as measured along comb teeth thereof was 520 μm, and a dimension L2 of the interdigital electrode structure as measured perpendicularly to the comb teeth thereof was 500 μm. In addition, an electrically insulating film (insulating film 9) was further provided over the base member 6 to cover the first electrode 51 and the second electrode 52. Through the insulating film 9, strip-shaped openings 70, each having a width of 5 μm, were provided as shown in FIG. 2 to overlap with the first electrode 51 and the second electrode 52. A dimension L3 as measured between respective center lines of the openings 70 shown in FIG. 2 was 60 μm. Furthermore, each of the samples of the gas adsorbent 1 was provided over the base member 6 to cover the insulating film 9 and have a thickness of 1 μm. This brought the gas adsorbent 1 into contact with the first electrode 51 and the second electrode 52 through the openings 70. The diameter D1 of the gas adsorbent 1 shown in FIG. 2 was 900 μm. In addition, the gas sensor 10 was further provided with a first terminal 81 extended from one end of the first electrode 51 to protrude out of the gas adsorbent 1 and a second terminal 82 extended from one end of the second electrode 52 to protrude out of the gas adsorbent 1.

The gas sensor 10 was placed in a nitrogen gas flow with a constant voltage applied between the first terminal 81 and the second terminal 82 and then nonanal was added to the gas flow to have a concentration of 1 ppm by volume. In this manner, each of the samples was exposed to the gas flow including nonanal until the electrical resistance value of each sample substantially stopped varying. The electrical resistance value of each sample was calculated based on the result of measurement of an electric current flowing between the first terminal 81 and the second terminal 82.

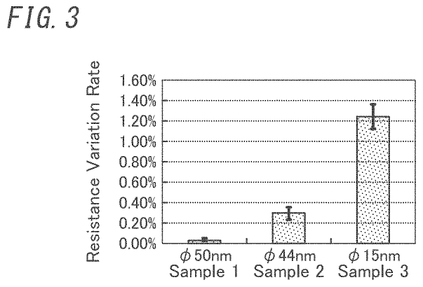

FIG. 3 shows the variation rates of electrical resistance values of the respective samples with respect to the electrical resistance value in the nitrogen gas flow. As can be seen easily from these results, the variation rate of the electrical resistance value increased more significantly in Sample #2 with a mean particle size of 44 nm than in Sample #1 with a mean particle size of 50 nm, and the variation rate of the electrical resistance values increased much more significantly in Sample #3 with a mean particle size of 15 nm than in Sample #1 with a mean particle size of 50 nm.

In this manner, the effect of the particle size of the conductive particles 21 on the sensitivity was confirmed.

3. Forming Samples

Silica particles with a mean particle size of 10 nm were provided as the insulating particles 3. As the conductive particles 21, particles of carbon black with a mean particle size of 15 nm were provided. As the solvent, dimethylformamide was provided. As the organic material 22, polyethylene glycol was provided.

The insulating particles 3, the conductive particles 21 and the organic material 22 were added to the solvent and stirred up, thereby preparing a mixed solution including the insulating particles 3 at a concentration of 10 mg/ml, the conductive particles 21 at a concentration of 10 mg/ml, and the organic material 22 at a concentration of 10 mg/ml.

Next, the mixed solution was applied by inkjet method to form a formed product in the shape of a film. This formed product was subjected to heat treatment at 50° C. for 20 minutes, thereby volatilizing the solvent from the formed product.

In this manner, Sample #4 was obtained as a gas adsorbent 1. In addition, Sample #5 was obtained as another gas adsorbent 1 in the same way as Sample #4 except that the mean particle size of the silica particles was changed into 30 nm. Furthermore, Sample #6 was obtained as still another gas adsorbent 1 in the same way as Sample #4 except that the mean particle size of the silica particles was changed into 100 nm. Furthermore, Sample #7 was obtained as yet another gas adsorbent 1 in the same way as Sample #4 except that the mean particle size of the silica particles was changed into 500 nm. Furthermore, Sample #8 was obtained as yet another gas adsorbent 1 in the same way as Sample #4 except that the mean particle size of the silica particles was changed into 1500 nm.

3. Evaluation Tests

The following evaluation tests were carried out on Samples #4-#8 described above and Sample #3 that was made to confirm the effect of the particle size of the conductive particles 21.

3-1. Micrograph Observation

The respective surfaces of these samples and cross sections, taken along their planes aligned with thickness direction, of these samples were observed through a scanning electron microscope (SEM). As a result, no voids 11 were recognized in Sample #3 containing no insulating particles 3 and in Samples #4 and #5 containing insulating particles 3 with a mean particle size of 10 nm and insulating particles 3 with a mean particle size of 30 nm, respectively. On the other hand, voids 11 were recognized in all of Samples #6-#8, each containing insulating particles 3 with a mean particle size of 100 nm or more.

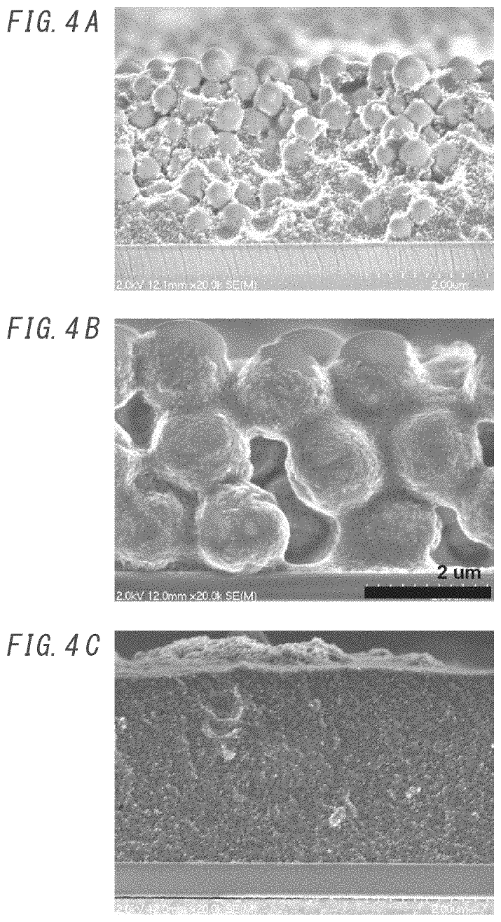

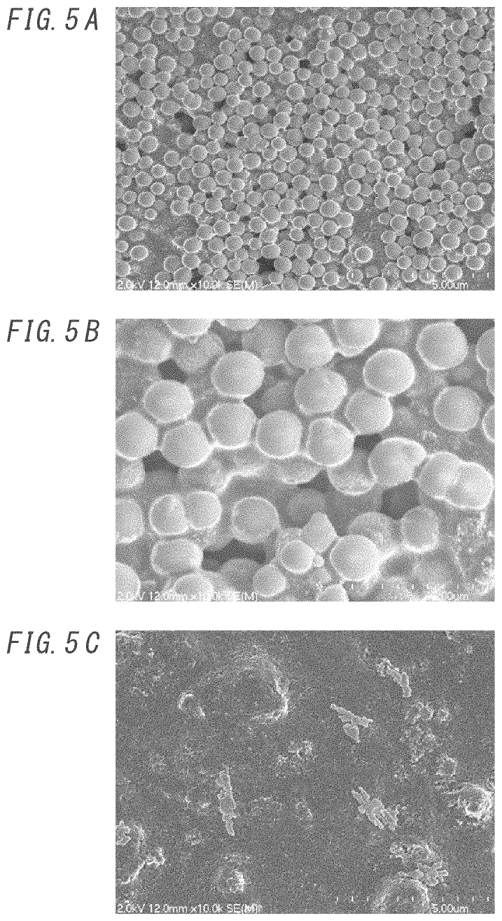

For your reference, cross-sectional micrographs of Sample #7 (containing insulating particles 3 with a mean particle size of 500 nm), Sample #8 (containing insulating particles 3 with a mean particle size of 1500 nm), and Sample #3 (containing no insulating particles 3) are shown in FIGS. 4A, 4B, and 4C, respectively, and their surface micrographs are shown in FIGS. 5A, 5B, and 5C, respectively. As can be seen from these micrographs, no voids 11 were recognized in Sample #3, while it was observed in Samples #7 and #8 how the adsorbing portions 2 adhered to the insulating particles 3 and voids 11 were created to form the porous structure. Note that in the cross-sectional micrograph of Sample #7 shown in FIG. 4A, the presence of voids 11 is still recognizable, even though their presence is not as clearly seen as in Sample #8 shown in FIG. 4B, because peeling occurred in the cross section.

As for Samples #7 and #8, the diameters of the voids 11 were measured in the following manner. Specifically, each of Samples #7 and #8 was cut off to expose their cross section. The cross section thus exposed was polished, and then observed through an electron microscope to capture the micrographs. Then, circles inscribed to the respective profiles of voids 11, appearing on the micrograph, were drawn and the diameter of the largest one of those inscribed circles was measured. For example, the diameter values of inscribed circles were measured with respect to ten voids 11 and the average of six intermediate ones of the diameter values, selected from the ten diameter values with the two largest ones and the two smallest ones excluded, was calculated and regarded as the diameter of the voids 11. As a result, the diameter of the voids 11 turned out to be 191 nm in Sample #7 and 684 nm in Sample #8.

3-2. Evaluation of Sensor Characteristics

The sensor characteristic of each sample was confirmed by the following method using a gas sensor 10 having the same configuration as the one described in the “1. Confirmation of effect of particle size of conductive particles” section.

The gas sensor 10 was placed in a nitrogen gas flow with a constant voltage applied between the first terminal 81 and the second terminal 82 of the sensor and then benzaldehyde was added for about 5 seconds to the gas flow to have a concentration of 1 ppm by volume. During this process, the amount of electric current flowing between the first terminal 81 and the second terminal 82 was measured. The electrical resistance value of each sample as a gas adsorbent 1 was calculated based on the result of the measurement.

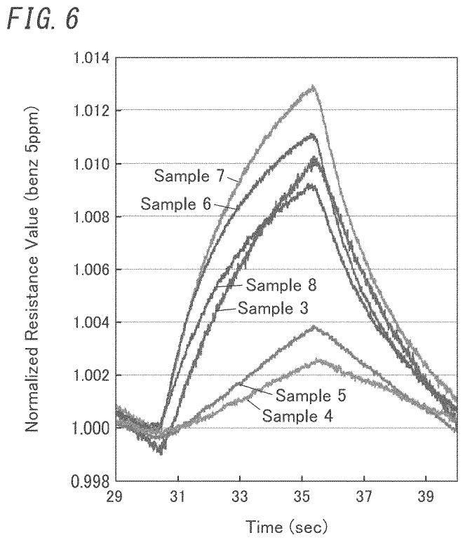

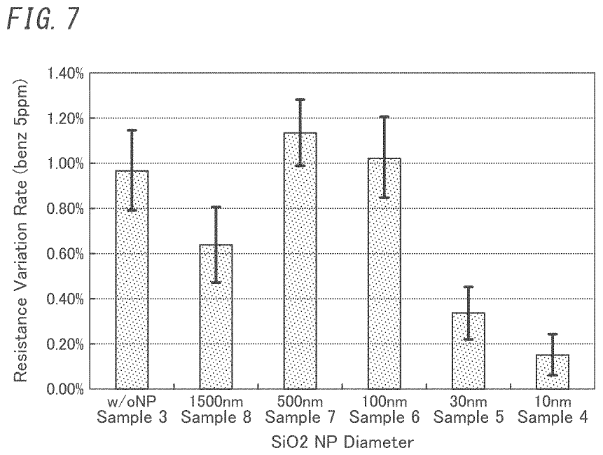

FIG. 6 shows how the electrical resistance values of the respective samples varied with time. In FIG. 6, the abscissa indicates the time elapsed. During a period from a point in time indicated as 30 plus seconds on the scale of the abscissas to a point in time indicated as 35 plus seconds there, benzaldehyde was added to the gas flow. On the other hand, the ordinate indicates the normalized electrical resistance values of the respective samples. Note that the normalized electrical resistance value was defined by regarding the electrical resistance value of each sample as measured in advance in the nitrogen gas flow as unity. FIG. 7 shows the average of the normalized electrical resistance values of the respective samples as measured when 5 seconds passed since benzaldehyde started to be added to the gas flow, in a situation where this test was carried out three times.

The results shown in FIG. 6 reveal that in each of these samples, the electrical resistance value started to increase at a point in time when benzaldehyde was added to the gas flow and decreased when benzaldehyde was no longer added to the gas flow.

Among these samples, in Samples #4 and #5 which contained insulating particles 3 with a mean particle size of 10 nm and insulating particles 3 with a mean particle size of 30 nm, respectively, and in which no voids 11 were recognized, the electrical resistance value did not increase as quickly as (i.e., exhibited lower responsivity than) Sample #3 containing no insulating particles 3.

On the other hand, in Samples #6-#8, each containing insulating particles 3 with a mean particle size of 100 nm or more, when benzaldehyde was added to the gas flow, their electrical resistance value increased more quickly than in Sample #3. Particularly in Sample #6 containing insulating particles 3 with a mean particle size of 100 nm and Sample #7 containing insulating particles 3 with a mean particle size of 500 nm (in Sample #7 among other things), the variation rate of the electrical resistance value at a point in time when 5 seconds passed was higher than in any other sample. Thus, it was confirmed that Samples #6 and #7 exhibited high sensitivities.

As can be seen from the foregoing description of an exemplary embodiment and examples, a gas adsorbent (1) according to a first aspect of the present disclosure includes an insulating particle (3), a plurality of conductive particles (21), and an organic material (22). The plurality of conductive particles (21) and the organic material (22) adhere to a surface of the insulating particle (3) to form an adsorbent particle (12). A plurality of such adsorbent particles (12) are aggregated together to form a porous structure.

The first aspect provides a gas adsorbent (1) which contains an organic material (22) and a plurality of conductive particles (21) and which allows an electrical resistance value to vary responsively upon exposure to a gas.

In a gas adsorbent (1) according to a second aspect of the present disclosure, which may be implemented in conjunction with the first aspect, a mean particle size of the insulating particle (3) is larger than a mean particle size of the plurality of conductive particles (21).

The second aspect increases the chances of the gas adsorbent (1) having a porous structure.

In a gas adsorbent (1) according to a third aspect of the present disclosure, which may be implemented in conjunction with the first or second aspect, the mean particle size of the insulating particle (3) is three times or more as large as the mean particle size of the plurality of conductive particles (21).

The third aspect increases the chances of the gas adsorbent (1) having a porous structure.

In a gas adsorbent (1) according to a fourth aspect of the present disclosure, which may be implemented in conjunction with any one of the first to third aspects, a diameter of a void (11) created in the porous structure is larger than the mean particle size of the plurality of conductive particles (21).

The fourth aspect increases the chances of the gas adsorbent (1) having a porous structure.

In a gas adsorbent (1) according to a fifth aspect of the present disclosure, which may be implemented in conjunction with any one of the first to fourth aspects, the plurality of conductive particles (21) and the organic material (22) adhere, in a shape of a film, onto the surface of the insulating particle (3).

The fifth aspect allows the gas adsorbent (1) to adsorb a gas particularly easily.

In a gas adsorbent (1) according to a sixth aspect of the present disclosure, which may be implemented in conjunction with any one of the first to fifth aspects, the organic material (22) contains a polymer.

The sixth aspect allows the gas adsorbent (1) to have heat resistance.

In a gas adsorbent (1) according to a seventh aspect of the present disclosure, which may be implemented in conjunction with any one of the first to sixth aspects, the plurality of conductive particles (21) includes a carbon material.

The seventh aspect particularly significantly increases the chances of the gas adsorbent (1) causing a variation in its electrical resistance value upon exposure to a gas.

In a gas adsorbent (1) according to an eighth aspect of the present disclosure, which may be implemented in conjunction with any one of the first to seventh aspects, a mean particle size of the plurality of conductive particles (21) falls within a range from 10 nm to 30 nm.

The eighth aspect increases the chances of the gas adsorbent (1) exhibiting increased sensitivity when adsorbing a chemical substance.

In a gas adsorbent (1) according to a ninth aspect of the present disclosure, which may be implemented in conjunction with any one of the first to eighth aspects, a mean particle size of the insulating particle (3) falls within a range from 100 nm to less than 1500 nm.

The ninth aspect increases the chances of the gas adsorbent (1) exhibiting increased responsivity when adsorbing a chemical substance.

A gas adsorbing device (20) according to a tenth aspect of the present disclosure includes: the gas adsorbent (1) according to any one of the first to ninth aspects; and a base member (6). Each of the plurality of adsorbent particles (12) of the gas adsorbent (1) includes: the insulating particle (3); a first coating layer (23) formed of the plurality of conductive particles (21) and coating the surface of the insulating particle (3) continuously; and a second coating layer (24) formed of the organic material (22) and coating a surface of the first coating layer (23) continuously. The porous structure is formed by connecting the plurality of adsorbent particles (12) with each other continuously and creating a void (11) surrounded with adjacent ones of the plurality of adsorbent particles (12). The gas adsorbent (1) is in contact with the base member (6) on at least one of the first coating layer (23) or the second coating layer (24).

The tenth aspect provides a gas adsorbing device (20) including a gas adsorbent (1) which contains an organic material (22) and a plurality of conductive particles (21) and of which the electrical resistance value varies responsively upon exposure to a gas.

A gas sensor (10) according to an eleventh aspect of the present disclosure includes: either the gas adsorbent (1) according to any one of the first to ninth aspects or the gas adsorbing device (20) according to the tenth aspect; and an electrode (5) electrically connected to the gas adsorbent (1).

The eleventh aspect provides a gas sensor (10) including a gas adsorbent (1) which contains an organic material (22) and a plurality of conductive particles (21) and of which the electrical resistance value varies responsively upon exposure to a gas.

A method of making a gas adsorbent (1) according to a twelfth aspect of the present disclosure includes: preparing a mixed solution containing an organic material (22), a plurality of conductive particles (21), an insulating particle (3), and a solvent; forming a formed product out of the mixed solution; and volatilizing the solvent out of the formed product.

The twelfth aspect allows making a gas adsorbent (1) which contains an organic material (22) and a plurality of conductive particles (21) and of which the electrical resistance value varies responsively upon exposure to a gas.

A method of making a gas adsorbent (1) according to a thirteenth aspect of the present disclosure, which may be implemented in conjunction with the twelfth aspect, includes forming the gas adsorbent (1) into a film shape by forming a film of the formed product.

The thirteenth aspect allows the gas adsorbent (1) to adsorb a chemical substance in a gas more easily by increasing the specific surface area of the gas adsorbent (1).

### REFERENCE SIGNS LIST

1 Gas Adsorbent
11 Void
12 Adsorbent Particle
2 Adsorbing Portion
21 Conductive Particle
22 Organic Material
23 First Coating Layer
24 Second Coating Layer
3 Insulating Particle
5 Electrode
10 Gas Sensor
20 Gas Adsorbing Device

1. A gas adsorbent comprising a plurality of adsorbent particles, the plurality of adsorbent particles being aggregated together to form a porous structure,
each of the plurality of adsorbent particles including: an insulating particle; and a plurality of conductive particles and an organic material, all of which adhere to a surface of the insulating particle.

2. The gas adsorbent of claim 1, wherein
a mean particle size of the insulating particle is larger than a mean particle size of the plurality of conductive particles.

3. The gas adsorbent of claim 2, wherein
the mean particle size of the insulating particle is three times or more as large as the mean particle size of the plurality of conductive particles.

4. The gas adsorbent of claim 1, wherein
a diameter of a void created in the porous structure is larger than the mean particle size of the plurality of conductive particles.

5. The gas adsorbent of claim 1, wherein
the plurality of conductive particles and the organic material adhere, in a shape of a film, onto the surface of the insulating particle.

6. The gas adsorbent of claim 1, wherein
the organic material contains a polymer.

7. The gas adsorbent of claim 1, wherein
the plurality of conductive particles includes a carbon material.

8. The gas adsorbent of claim 1, wherein
a mean particle size of the plurality of conductive particles falls within a range from 10 nm to 30 nm.

9. The gas adsorbent of claim 1, wherein
a mean particle size of the insulating particle falls within a range from 100 nm to less than 1500 nm.

10. A gas adsorbing device comprising: the gas adsorbent of claim 1; and a base member,
each of the plurality of adsorbent particles of the gas adsorbent includes:
the insulating particle;
a first coating layer formed of the plurality of conductive particles and coating the surface of the insulating particle continuously; and
a second coating layer formed of the organic material and coating a surface of the first coating layer continuously,
the porous structure being formed by connecting the plurality of adsorbent particles with each other continuously and creating a void surrounded with adjacent ones of the plurality of adsorbent particles,
the gas adsorbent being in contact with the base member on at least one of the first coating layer or the second coating layer.

11. A gas sensor comprising:
the gas adsorbent of claim 1; and
an electrode electrically connected to the gas adsorbent.

12. A gas sensor comprising:
the gas adsorbing device of claim 10; and
an electrode electrically connected to the gas adsorbent.

13. The gas adsorbent of claim 2, wherein
a diameter of a void created in the porous structure is larger than the mean particle size of the plurality of conductive particles.

14. The gas adsorbent of claim 13, wherein
the mean particle size of the insulating particle is three times or more as large as the mean particle size of the plurality of conductive particles.

15. The gas adsorbent of claim 5, wherein
the organic material contains a polymer.

16. The gas adsorbent of claim 7, wherein
a mean particle size of the plurality of conductive particles falls within a range from 10 nm to 30 nm.

17. The gas adsorbent of claim 2, wherein
the plurality of conductive particles and the organic material adhere, in a shape of a film, onto the surface of the insulating particle.

18. The gas adsorbent of claim 17, wherein
the plurality of conductive particles includes a carbon material.

19. The gas adsorbent of claim 2, wherein
the plurality of conductive particles includes a carbon material.

20. The gas adsorbent of claim 5, wherein
the plurality of conductive particles includes a carbon material.
